SEQUENCE LISTING (1 sequence)

SEQ ID NO: 1 - protein, 8 aa, Vaccinia virus
  PEPTIDE (1)..(8)
DSNFFTEL

(19) United States
(12) Patent Application Publication
(10) Pub. No.: US 2007/0160571 A1
(43) Pub. Date: Jul. 12, 2007

(54) Composition and method for inducing protective vaccine response

(76) Inventor: Mitchell Krathwohl, Indianapolis, IN (US)

Correspondence Address: DONNA J. RUSSELL, 1492 ANTHONY WAY, MT. JULIET, TN, 37122, US

(21) Appl. No.: 11/329,655

(22) Filed: Jan. 11, 2006

(51) Int. Cl. A61K 39/295; A61K 38/19

(52) U.S. Cl. 424/85.1; 424/202.1

(57) ABSTRACT

A method for inducing a protective immune response is disclosed, the method utilizing a composition comprising a CXCR3-binding chemokine such as CXCL10 (IP-10) administered in conjunction with a protein, peptide, polynucleotide, or other target antigen.

### STATEMENT IN REGARD TO GOVERNMENT RIGHTS

This invention was made in part with funding provided by the United States Government Grant number U56 AI 57164 awarded by the National Institutes of Health). The U.S. Government may therefore have certain rights in this invention.

### FIELD OF THE INVENTION

The present invention relates to compositions and methods for preparing vaccines. More particularly, the invention relates to compositions and methods for generating immune responses to protein, peptide, or other subunit vaccines.

### BACKGROUND OF THE INVENTION

Vaccines are important for prevention of a variety of diseases, and can be particularly effective for prevention of viral disease. Certain immunological principles govern vaccine efficacy, but these principles are not well understood. Generally, infection with a wild-type pathogenic virus will produce long-lasting immunity that protects against illness when the host is re-exposed to the same virus. This, of course, is only of benefit if the host survives the initial infection with the pathogenic virus. In some cases, live virus such as the vaccinia virus vaccine used for smallpox can provide long-term protection but may also cause lymphadenopathy, fever, and life-threatening disease. Whole virus vaccination is not recommended for millions of people who may be at risk for vaccine complications because of heart disease, immune deficiencies, and conditions such as eczema or atopic dermatitis.

Depending upon the route of immunization, the immunogen formulation, and the use of adjuvants in specific vaccines, immune induction can be manipulated to favor either T_{H}1 (cellular) or T_{H}2 (antibody) responses. For viral infections, both cellular and antibody responses may be involved in immunity, but the relative role of each may vary, depending on the type of virus. For vaccinia virus, for example, the T_{H}1 response predominates in mice when live virus is used to immunize, while the T_{H}2 response predominates when outer membrane proteins of the virus are used as vaccine (Fogg, et al., J. Virol. (2004) 78: 10230-10237).

For many diseases, cell-mediated immunity or local mucosal immunity is more important for early protection than is the antibody response. Cellular immune responses produce large numbers of effector cells in a relatively short time, while the antibody response develops more slowly. Virus-specific CD8 CTLs generally appear about one week after acute viral infection, and their numbers rapidly increase to peak at 2-3 weeks after infection. The peak often corresponds to the period when the virus is being cleared by the host. CD8 cells exert their effects through two main mechanisms: direct attack on virus-infected cells and secretion of interleukins (ILs) and cytokines such as IFN-γ, TNF-α, and IL-2 that may also play a role in clearing virus-infected cells.

Immune induction efficiency is increased if immunogen (antigen) is presented by antigen-presenting cells (APCs) such as macrophages and dendritic cells. Immature dendritic cells are derived from the same bone marrow precursors as macrophages. Dendritic cells (DCs) take up antigen, generate peptide epitopes from it, then load these epitopes into major histocompatibility complex (MHC) molecules for expression at the cell surface. When a dendritic cell takes up pathogenic organisms, it becomes activated, stimulating secretion of cytokines. If the DC fails to be activated, it induces tolerance to the antigens it bears. DC maturation therefore represents a key control point in the decision for immunity versus tolerance.

After encountering antigen in the context of a danger signal, DC undergo a program of maturation that enables them to efficiently induce an antigen-specific T cell immune response. A large diversity of danger signals have been defined that serve to promote DC maturation, including microbial constituents, cytokines, and UV light (Bell, et al., “Dendritic Cells,” Advances in Immunology (1999) 72: 255-324). DC also can amplify such danger signals by autocrine and paracrine release of cytokines such as interferon γ (IFN-γ), which has been shown to act as a natural adjuvant through its effects on DC maturation (Proietti, et al., J. Immunol. (2002) 169: 375-383). DC under appropriate stimulation can both secrete and respond to IFN-γ, but it is not clear how IFN-γ mediates DC maturation. DC maturation has been shown to involve members of the MAP kinase family of signaling proteins (Ardeshna, et al., Blood (2000) 96 (3): 1039-1046), yet IFN-γ has not been shown to induce this pathway in DC. However, interferons are known to induce expression of a number of proteins such as chemokines with downstream effector functions. Whether chemokines might work as downstream signals or amplifiers of maturation signals has remained in question.

Peptide vaccines provide an alternative to whole-virus vaccine and may pose less risk than do whole-virus vaccines. Whole-virus vaccine may, for example, pose a risk of transmission of the vaccine strain to unvaccinated individuals. Since the beginning of the United States Armed Forces smallpox vaccination program in December 2002, for example, there have been 30 reported cases of accidental contact transmission (MMWR Morb Mortal Wkly Rep, (U.S. Centers for Disease Control, 2004) 53: 103-105; Garde et al., JAMA (2004) 291: 725-727).

Protein and peptide vaccines, DNA vaccines, and other vaccine formulations provide alternatives to whole microbe vaccines that are less likely to produce unwanted side-effects and are more likely to be easier to produce. Peptides are poorly immunogenic in the absence of co-administered adjuvants. When epitopes are administered out of context of the whole antigen they may lack the ability to stimulate DC maturation and cytokine release. What is needed are compositions and methods for improving the immunogenicity and protective immune response provided by these vaccine formulations.

### SUMMARY OF THE INVENTION

The present invention provides a composition comprising a therapeutically effective amount of one or more CXCR-3 binding chemokines, such as human interferon-gamma inducible protein (IP-10, also known as CXCL10), to stimulate a protective immune response against future challenge with an antigen administered in conjunction with the CXCR-3 binding chemokine(s). The invention also provides a composition comprising a therapeutically effective amount of and at least one CXCR-3 binding chemokine, such as IP-10, and at least one immunogen to stimulate immune protection against future infection by an infectious agent. In one embodiment, the at least one immunogen is a live attenuated virus, an inactivated virus, a viral or bacterial protein, or a peptide derived from a viral or bacterial protein. Proteins and peptides can also comprise modified proteins and peptides that differ from the wild-type protein or peptide by amino-acid substitution or other modification, particularly those modifications that may decrease degradation of the protein or peptide or increase its immunogenicity. DNA, peptide nucleic acids, or other immunogens may also comprise compositions of the invention.

In one embodiment, the invention comprises a vaccine comprising an immunogenic amount of at least one antigen chosen from among at least one bacterial antigen, at least one viral antigen, at least one fungal antigen, at least one tumor antigen, or a combination thereof, an of at least one CXCR3-binding chemokine effective to induce maturation of lymph-node derived dendritic cells.

The invention also provides a method for stimulating a Th1-type immune response to an antigen which can be administered in a vaccine. In the method of the invention, a CXCR3-binding chemokine, such as, for example, IP-10 or I-TAC, is administered to stimulate dendritic cells (DC) in the tissues to mature and promote the development of a Th1-type immune response to one or more immunogenic compositions or antigens when administered as a vaccine in conjunction with administration of at least one CXCR-3 binding chemokine.

The invention also provides a method for inducing maturation of dendritic cells to shift the immune response to antigen toward a Th1-type response by administering a therapeutically effective amount of chemokine IP-10 sufficient to stimulate dendritic cell maturation in the tissues in which the antigen is delivered. In one embodiment, the invention provides a method for inducing a Th1-type response to peptide and protein antigens that might most commonly induce a Th2-type immune response.

The invention also provides peptides comprising the amino acid sequence DSNFFTEL for use in subunit vaccines.

### BRIEF DESCRIPTION OF THE DRAWINGS

FIG. 1 is a series of photographs of microscopy illustrating that CXCL10 induces morphologic and phenotypic changes in DC characteristic of maturation. Photographs illustrate results of treatment as follows: Control—1a; LPS—1b; IP-10—1c; MIP-3-beta—1d. Treatment of monocyte derived dendritic cells (MDDC) with CXCL10 causes cells to develop extensive dendritic processes (1c), similar to LPS-treated cells (1b).

FIG. 2 is a graph illustrating that lymph node-derived dendritic cells (LNDC) treated with CXCL10 for 48 hours produce IL-12 by ELIspot assay as shown by the increased number of spots formed as compared to control.

FIG. 3 is a graph of the proliferation index illustrating that DC cultured with CXCL10 become potent stimulators of T cells. Allogeneic T cells were cultured in various ratios with MDDC that had been grown with or without CXCL10. After 7 days, the total number of cells was determined and expressed as a ratio of the number of input cells. The proliferation index is indicated on the Y axis and the ratio of T-cells to dendritic cells is indicated on the X axis. Each DC ratio was determined in triplicate.

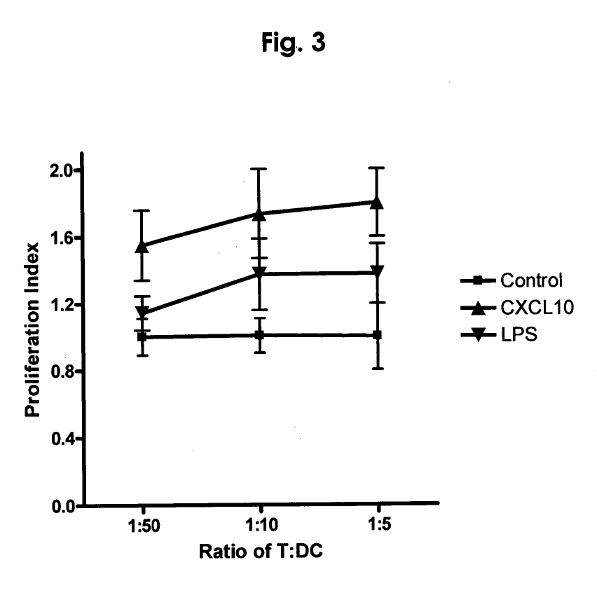

FIG. 4 is a graph of the proliferation index following allogeneic T-cell stimulation by co-culture with CD34-derived DC, at the ratios indicated on the X axis, that had been grown with or without CXCL10. After 7 days, the total number of cells was determined and expressed as a ratio of the number of input cells. The proliferation index is indicated on the Y axis and the ratio of T-cells to dendritic cells is indicated on the X axis. Each DC ratio was determined in triplicate.

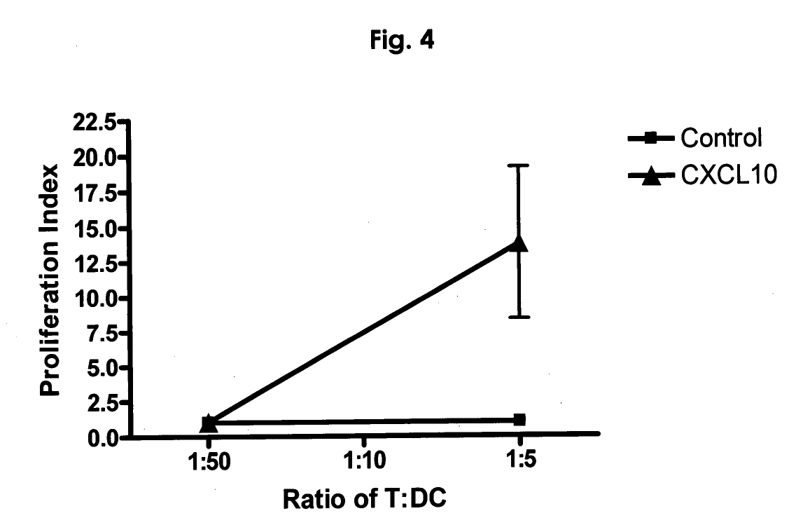

FIG. 5 shows survival diagrams of treated DC. LNDC were cultured without growth factors in the presence or absence of CXCL10. Cell viability was determined daily by trypan blue exclusion. All cultures were set up in triplicate.

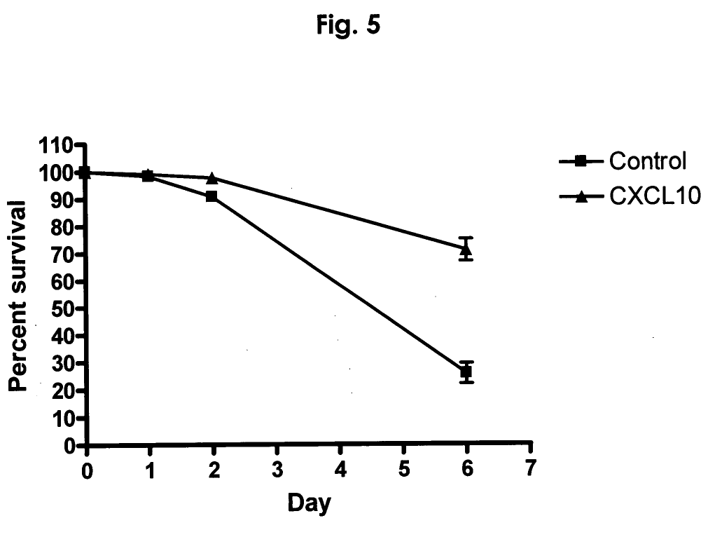

FIG. 6 is a series of photos showing the results of mice in several treatment groups injected with vaccinia virus. Only mice in the CXCL10+ peptide group were protected from infection as shown by the lack of tail ulcers.

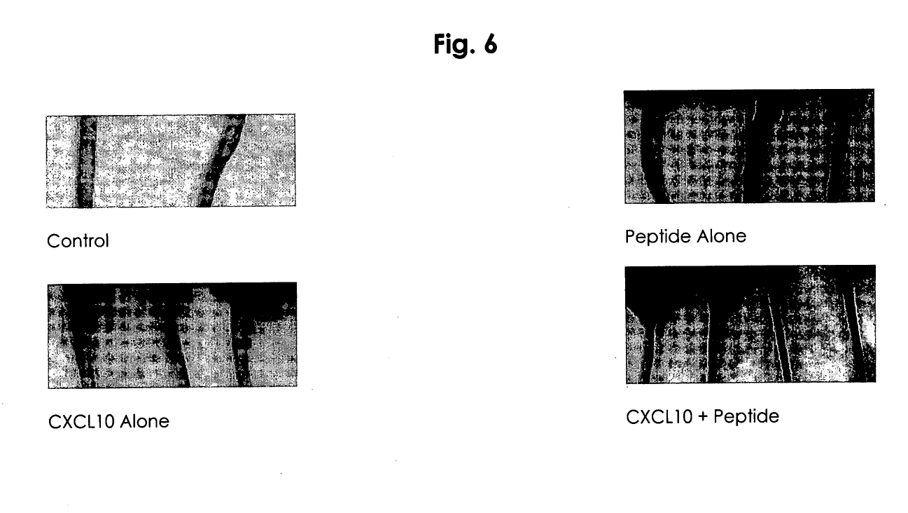

FIG. 7 is a bar graph of the quantitative results of mice injected with vaccinia virus in each of several treatment groups. Mice were injected twice with CXCL10 with or without vaccinia peptide, with peptide alone or normal saline alone. Four weeks after the first injection, all mice were injected with vaccinia virus intradermally at the base of the tail and the development of tail ulcers was monitored. Ulcer severity was scored on a 0 to 4+ scale for each group. The graph shows quantitative counts of tail ulcer formation.

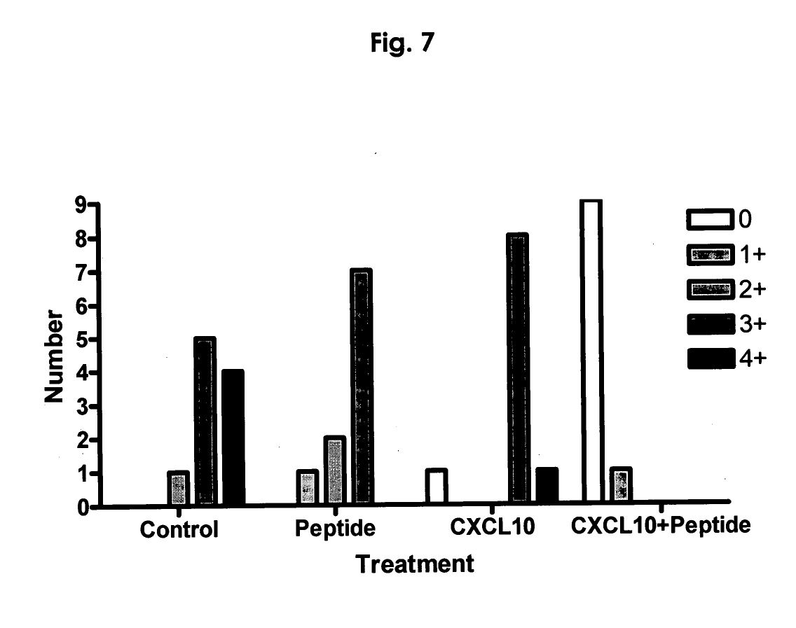

FIG. 8 is a bar graph illustrating induction of peptide-specific cytotoxic T cells by CXCL10 administration with a vaccinia-derived peptide. Spleens were removed from all groups of mice, and splenocytes were tested for proliferation against the injected vaccinia peptide. After 5 days, the total number of cells was enumerated using a fluorescence-based method for each group. The stimulation index is indicated on the Y axis and the treatment group is indicated on the X axis.

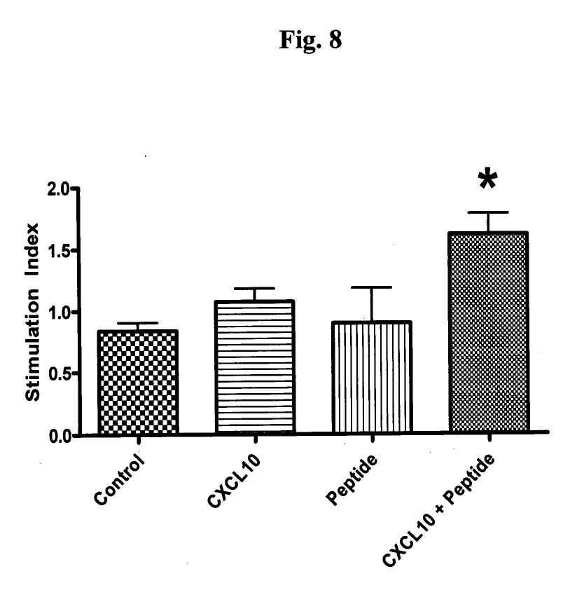

FIG. 9. is a line graph illustrating the percent of specific lysis when splenocytes were stimulated with peptide for 5 days, and then incubated with target cells that had been pulsed with peptide and labeled with the fluorescent tracer BCECF. Specific lysis was calculated for each treatment group. The ratio of effector to target cells is indicated on the X axis and the percentage of specific lysis is indicated on the Y axis.

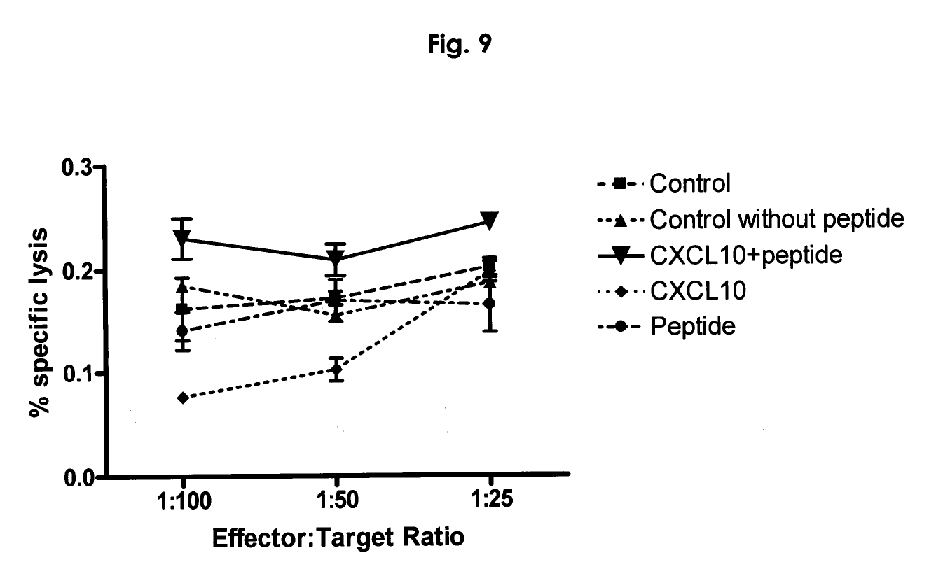

### DETAILED DESCRIPTION

The present invention provides a composition comprising a therapeutically effective amount of a CXCR3-binding chemokine and at least one immunogen for administration as a vaccine to stimulate immune protection against future infection by an infectious agent. In one embodiment, the CXCR3-binding chemokine is interferon-inducible protein (IP-10). The invention also provides a method for stimulating a Th1-type immune response to an antigen which can be administered in a vaccine. Furthermore, the invention provides a method for inducing maturation of dendritic cells to promote a cellular immune response to antigen by administering a therapeutically effective amount of a CXCR3-binding chemokine such as, for example, IP-10, I-TAC, or Mig sufficient to stimulate dendritic cell maturation in the tissues in which the antigen is delivered. The invention also provides a method for inducing a Th1-type response to peptide and protein antigens that might most commonly induce a Th2-type immune response. The invention also provides a method for inducing IL-12 production by dendritic cells.

It is to be understood that functional moieties of CXCR3-binding chemokines such as IP-10 and I-TAC, for example, may also be used in the composition and method of the present invention. Peptides and protein-binding domains have been shown to retain much of the functionality of a full-length protein when their sequences are isolated from that of the full-length protein itself.

The inventors tested the effects of chemokine CXCL10, also known as IP-10, on DC maturation in monocyte-derived, CD34 hematopoietic progenitor cell-derived, and immature lymph node DC and found that CXCL10 promoted the up-regulation of markers typical of maturation in all of the DC from these various sources. CXCL10 also induced IL-12 production in these DC, and enabled DC to form conjugates with T cells and stimulate T cell proliferation and interferon-γ production. The inventors also demonstrated that survival of DC is increased by CXCL10 administration. Furthermore, the inventors demonstrated that IP10-induced maturation of dendritic cells stimulated a Th1-type immune response. By administering a vaccine comprising a combination of antigen and IP-10, the inventors were able to induce a CD8 T cell response and to generate a protective response against viral challenge. A peptide vaccine according to the present invention was used by the inventors, for example, to protect mice from experimental challenge with vaccinia infection.

CXCL10, as well as inducible T cell-α chemoattractant (I-TAC, CXCL11) and monokine-induced by γ-interferon (Mig, CXCL9) are chemokines that control leukocyte migration via their binding to chemokine receptor CXCR3. Myeloid dendritic cells, such as those that are induced toward maturation in the method of the present invention, are not known to express the CXCR3 receptor.

Typically, vaccines are prepared for injection into a human or mammalian subject. Injectable vaccines can be prepared as liquid solutions or suspensions. Solid forms can be prepared which are suitable for solution in, or suspension in, liquid prior to injection. The preparation may also be emulsified. The active immunogenic ingredient is often mixed with a pharmaceutically acceptable carrier which is compatible with the active ingredient. Suitable carriers include, but are not limited to, water, dextrose, glycerol, saline, ethanol, and combinations thereof. The vaccine may contain additional agents such as wetting or emulsifying agents, pH buffering agents, or adjuvants which enhance the effectiveness of the vaccine.

Vaccines may be conventionally administered parenterally using subcutaneous or intramuscular injection. Other modes of administration may include oral administration, nasal administration, rectal administration, and vaginal administration, which may involve combining the immunogen with pharmaceutically acceptable carriers such as mannitol, lactose, starch, magnesium stearate, sodium saccharine, cellulose, magnesium carbonate, or other carrier. Compositions for oral administration may form solutions, suspensions, tablets, pills, capsules, sustained release formulations or powders. A vaccine of the present invention can be administered by enteric-coated capsule for release of the polypeptide into the lumen of the intestine. Vaccines may be delivered intravenously, although IV administration may be associated with an increased risk of side-effects. Vaccines may be inhaled, and antigens may be pegylated (attached to at least one polyethylene glycol moiety) to increase the half-life of the antigen in the tissues.

Peptide or other immunogen may be formulated into the vaccine as neutral or salt forms. Pharmaceutically acceptable salts include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups may also be derived from inorganic bases such as, for example, sodium, potassium ammonium, calcium, or ferric hydroxides, and such organic bases as isopropylamine, trimethylamine, 2-ethylamino ethanol, and histidine.

Vaccine is administered in a manner compatible with the dosage formulation, and in such amount as will be therapeutically effective and immunogenic. The quantity to be administered depends on the subject to be treated, taking into account, for example, the subject's age and overall health, and the degree of protection desired. Precise amounts of active ingredient (peptide immunogen) to be administered depend on the judgment of the practitioner. Suitable dosage ranges generally require several hundred micrograms of active ingredient per vaccination. Also variable are regimes for initial administration and booster vaccinations, which should be determined by the judgment of the practitioner. Dosage of vaccine will depend on the route of administration and will vary according to the size of the host.

Adjuvants for use in combination with immunogen for vaccination include, but are not limited to, aluminum hydroxide or phosphate, also known as alum, commonly used as 0.05 to 0.1 percent solution; aggregation of the protein in the vaccine by heat treatment with temperatures ranging between 70° C. for 30 seconds to 101° C. for 2 minutes.

Compositions of the present invention can comprise vials containing a combination of one or more antigens comprising, for example, whole virus, proteins, or peptides, and IP-10. Separate vials containing, individually, IP-10 and one or more antigenic compositions are also provided by the invention. The invention provides compositions for delivery of vaccine “cocktails” which can increase the cellular immune response to one or more infectious agents.

In the method of the invention, IP-10 can be administered concurrently with the target antigen or antigens. IP-10 may also be administered within an effective time prior to the administration of antigen or within an effective time following administration of antigen. IP-10 may therefore be part of a vaccine administered to a subject or may be an immunotherapeutic agent delivered prior to or after vaccination in order to stimulate the subject's immune system to generate a Th1-type response to the infectious agent to which the vaccine is directed.

The invention provides compositions and methods for administering vaccines for bacterial and viral infectious agents, such as, for example, human immunodeficiency virus (HIV), vaccinia virus (for smallpox vaccine), herpes simplex virus (HSV), influenza virus, Ebola virus, Dengue virus, and SARS-CoV. In one embodiment, a vaccine is prepared using a polypeptide comprising SEQ ID NO: 1. It is to be understood that such a polypeptide may comprise additional amino acids at the C-terminus or N-terminus, and that functionally equivalent amino acid substitutions may be made to the amino acid sequence described by SEQ ID NO: 1. Such modifications are within the scope of one of skill in the art, provided the present disclosure, and are within the scope of the invention as described. Such a polypeptide may be provided through various means and in combination with other antigenic compositions, adjuvants, and other agents. Polypeptides of the invention may also be provided in the form of nucleic acids encoding the polypeptides, from which the polypeptide may be expressed. Given the polypeptide sequence, it is well within the expertise of one of skill in the art to predict nucleotide sequences that would encode such a polypeptide.

The invention also provides compositions and methods for administering vaccines containing tumor-associated antigens in order to generate a cellular immune response to tumor cells for the treatment of cancer. The immune system recognizes tumor-specific antigen(s), usually through the help of an APC such as a dendritic cells. APCs engulf tumor-specific antigens, break them into peptides and display peptide fragments on the surface of their own cells, complexed with major histocompatibility complex (MHC) proteins located on the surface of the APC. CD8+ cells bind to these complexes on the APC. Once activated, CD8+ cytotoxic T lymphocytes can migrate into tumor masses and cause cytolysis of tumor cells. A variety of tumor-specific antigens have been identified for specific types of cancer, such as prostate cancer and melanoma. In these cases, previously-identified antigens can be administered in conjunction with IP-10 to promote a cell-mediated immune response to tumor cells expressing the antigen. In other cases, however, specific antigens have not been identified. In these cases, a CXCR3-binding chemokine such as IP-10 can be administered in conjunction with acid-eluted peptides derived from autologous tumors. A procedure for obtaining these types of peptides has been described by Zitvogel et al. (J. Exp. Med. (1996) 183: 87-97).

Viral, bacterial, fungal, or tumor antigens, for example, may also be provided as one or more fusion peptides containing cell-permeable peptide sequences for delivery of the peptide antigen or antigens to the interior of a dendritic cell.

Additional cytokines or chemokines may also be provided in conjunction with a vaccine as provided by the present invention in order to increase or modulate the immune response to the antigen or antigens of choice in the vaccine. Many such immune-modulating cytokines and chemokines are known to those of skill in the art of immunology and vaccine development.

The invention may be further described by means of the following non-limiting examples.

### EXAMPLE 1

### Human

CXCL10 was purchased from Peprotech (Rocky Hill, N.J.) and certified endotoxin-free by the manufacturer. GM-CSF was a kind gift from Immunex Corporation. IL-4, TNF-α were also from Peprotech.

PBMCs from normal human donors were purchased from AllCells. PBMCs were incubated in 75 cm² flasks for 2 hours at 37° C. to adhere monocytes. Flasks were washed extensively with PBS to remove non-adherent cells. The remaining cells were removed by scraping and routinely consisted of >70% CD14+ cells. Resulting monocytes were cultured in 24 well plates at 5×10⁵ cells/ml in RPMI 1640+10% heat-inactivated FBS (Hyclone)+ antibiotics, and 100 ng/ml GM-CSF and IL-4. Cells were cultured in triplicate with or without the chemokine CXCL10 at 100 ng/ml added at the beginning of culture and replaced every 3-5 days, or added only at day 7 of culture. Cells were cultured for 6-9 days then either phenotyped, or used for functional assays described below.

Bone marrow-derived CD34+ cells were purchased from AllCells and were >95% CD34+. Cells were cultured at 5×10⁴ cells/ml in RPMI 1640+10% FBS with 100 ng/ml GM-CSF and 2.5 ng/ml TNF-α. Cells were cultured in triplicate either with or without the chemokine CXCL10 at 100 ng/ml added at day 0 and replenished every 3-5 days or else added only at day 7. Cells were harvested for further analysis on day 10-12.

Discarded human lymph node tissue that had been surgically removed for diagnostic purposes was recovered. Tissue was pathologically normal by routine hematoxylin and eosin stain. Lymph nodes were minced into small pieces and made into a single cell suspension by mechanical disruption with the barrel of a syringe. Lineage+ cells were depleted by 3 rounds of selection. The resulting cells were found to be >90% Lin-HLA-DR++ with >95% viability. The resulting DC were incubated at 5×10⁵ cells/ml in RPMI 1640+10% FBS with 100 ng/ml GM-CSF and either with or without CXCL10 at 100 ng/ml for 3 days. Cells were then harvested and analyzed.

Lymph node tissue was cut into small pieces 2×2×2 mm and cultured on Transwell insert supports in 24 wells plates, leaving the top surface exposed to air and the bottom surface resting on a membrane with 5 μm pores. The bottom wells contained RPMI 1640+10% FBS and antibiotics. Purified lymph node DC were cultured with or without CXCL10 (100 ng/ML) for 3 days, then labeled with a fluorescent tracking dye (Cell Tracker Green, CTG) according to the manufacturer's instructions and placed in bottom wells and allowed to migrate into the tissue through the pores. Slices were then harvested and either stained for confocal microscopy or made into single cell suspensions and analyzed for the presence of treated DC and T cell proliferation. For confocal microscopy, a fluorescent monoclonal antibody to CD3 was stained with slices while in the Transwell inserts for 2 hours. Slices were then placed in agar and analyzed by Confocal microscopy for the presence of CTG+ cells adjacent to CD3+ cells.

For flow cytometry, cells were washed, and then resuspended with various combinations of fluorescent monoclonal antibodies at 4° C. in the dark for 1 hour. Appropriate control antibodies of the same isotype with irrelevant specificity were used. Cells were then washed and analyzed on a FACscan flow cytometer (BD Biosciences). Percentages of positive cells were calculated using the FlowJo software package (TreeStar).

For IL-12 intracellular flow cytometry, surface antigen staining was performed, and cells were washed and placed in Cytofix/Cytoperm buffer (Pharmingen) containing saponin for permabilization. A fluorescent monoclonal antibody to IL-12 was incubated with cells in the dark at 4° C. After 1 hour, cells were washed and then analyzed for IL-12 expression by flow cytometry.

IL-12 Elispot was performed using purified LNDC added to the wells of an ELIspot plate (BD) and allowed to adhere to the PVDF membrane. CXCL10 (100 ng/ml) was added to some cultures of cells, and cells were incubated for 48 hours to allow IL-12 production. Plates were then harvested, and IL-12 was detected by a sandwich assay as per the manufacturer's instructions. Spots were counted visually. Each well was tested in triplicate, and the entire assay was performed at least twice with identical results.

Allogeneic T cells were purified from peripheral blood mononuclear cells by column purification for T cell proliferation assay. MDDC or CD34-derived DC were washed and mixed with T cells in the indicated ratios. Triplicate determinations were made for each ratio. Cells were cultured for 7 days, and the total number of cells was then counted using a hemocytometer. The entire experiment was performed at least twice with identical results.

Monocytes were cultured in RPMI1640+10% FBS and antibiotics with GM-CSF and IL-4 for 7 days for the intracellular signaling assay. Either normal saline or CXCL10 (100 ng/ml) was added to the cultures for 10 minutes, then cultures were placed on ice. Surface markers (CD11c and HLA-DR) were stained using monoclonal antibodies, then cells were fixed and permeabilized with Cytofix/Cytoperm. Fluorescent monoclonal antibodies to p-ERK, p-JNK or p-p38 were then added and cells were stained for 1 hour. Cells were then washed and analyzed by flow cytometry.

For the survival assay, lymph node derived cells (LNDC) were washed and grown in RPMI 1640+10% FBS but without addition of GM-CSF. CXCL10 was added to some cultures of LNDC set up in triplicate at 100 ng/ml. Cells viability was determined by trypan blue exclusion daily.

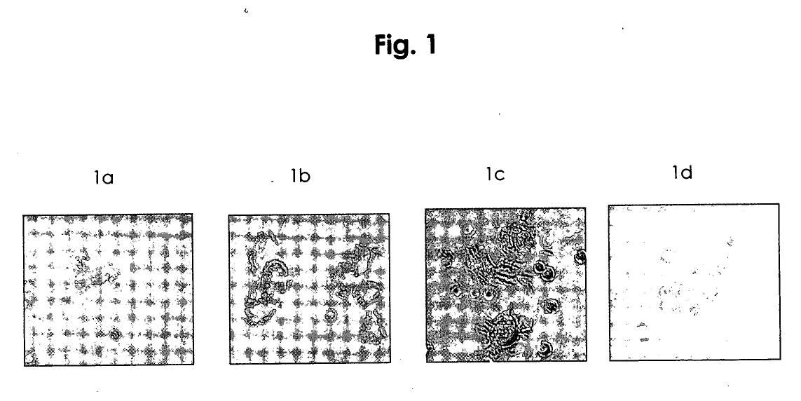

To determine whether CXCL10 would promote the maturation of DC, the inventors induced monocytes to differentiate into DC with GM-CSF and IL-4. CXCL10 was added to some cultures on the day of culture initiation and replaced every 3-5 days, and the resulting cells were analyzed after 7-9 days. DC treated with CXCL10 developed extensive dendritic processes compared to control cells (FIG. 1). In fact, CXCL10-treated cells appeared similar to cells matured with E. coli LPS. Next, the MDDC cultured with or without CXCL10 were analyzed for expression of DC markers typical of mature cells. CXCL10 treatment resulted in the upregulation of markers typical of maturation, including HLA-DR, CD83, CD40, CD80, and CD86. Flow cytometry indicated that DC treated with CXCL10 express IL-12. CXCL10 resulted in the down-regulation of the monocyte marker CD14. The inventors further tested the ability of CXCL10 to affect monocyte maturation when added only after 7 days of culture with GM-CSF and IL-4. CXCL10 increased expression of maturation markers HLA-DR, CD83, CD86 and CD80 but not CD40.

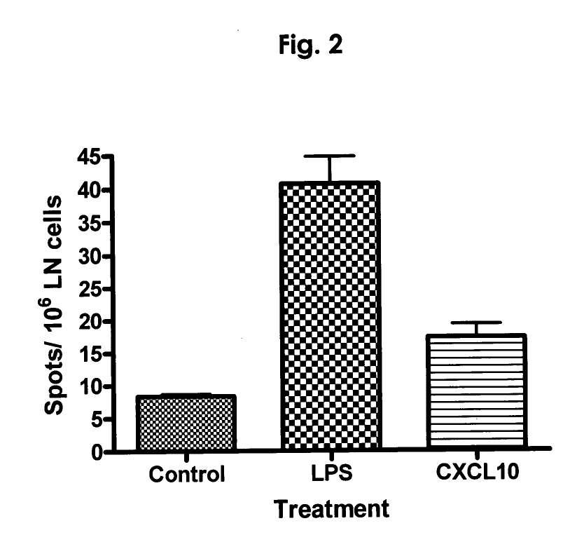

Mature DC produce IL-12 and IL-6. CXCL10 induced the expression of IL-12, in contrast to control cells or cells treated with the chemokine CCL19 (FIG. 2). Thus, CXCL10 appears to promote the full maturation of MDDC.

DC derived from CD34+ hematopoietic progenitors appear to resemble Langerhans cells as well as cells resembling interstitial DC, and may be better at stimulating CD8 T cells than MDDC. CD34+ hematopoietic progenitor cells were cultured with GM-CSF and TNF-α, either with or without CXCL10, for 7 days. Like MDDC, CXCL10 resulted in increased expression of maturation markers on these CD34-derived DC. Intracellular flow cytometry for IL-12 also confirmed that progenitors treated with CXCL10 were fully mature DC. Thus, CXCL10 also induces maturation of CD34-derived DC.

Pathologically normal lymph node tissue removed from patients for diagnostic purposes was used to determine whether CXCL10 would also induce maturation of uncultured immature DC. DC were purified by negative selection with magnetic beads and confirmed to be in primarily in the immature state. These LNDC were then cultured with or without CXCL10. CXCL10 induced expression of typical maturation markers on these cells as well. To determine whether these cells expressed IL-12, LN cells were cultured on ELIspot plates and determined the number of IL-12+ spots 48 hours later. CXCL10 treated cells produced significant levels of IL-12 over control cells, indicating that CXCL10 can also induce maturation in purified human immature DC.

To investigate whether CXCL10-matured DC would stimulate T cells, MDDC that had been treated with or without CXCL10 were cultured with various ratios of allogeneic T cells. After 7 days of co-culture, the total number of cells was counted in each well. MDDC that had been treated with CXCL10 significantly induced proliferation of T cells. CXCL10-treated DC appeared to be even better at stimulating allogeneic T cells compared to MDDC. To determine whether DC would induce T cell proliferation in lymph nodes, cultured slices of normal lymph node were placed on solid supports. DC purified from autologous lymph nodes were cultured with CXCL10 for 3 days, then labeled with a fluorescent dye and incubated in the bottom well of the inserts. Cells were allowed to migrate through the pores of the support into the lymph node slice. After 3 days, slices were harvested and stained with an anti-CD3 fluorescent antibody. The slice was subjected to confocal microscopy for analysis. DC treated with CXCL10 formed conjugates with T cells, while control DC did not. Slices were further analyzed by flow cytometry to investigate T cell proliferation. BrdU was added to slice cultures at the same time as the DC to label proliferating T cells. Purified DC were either treated with CXCL10 alone or CXCL10 and tetanus toxoid in order to stimulate toxoid-specific T cells. CXCL10-treated DC significantly induced proliferation of T cells as measured by BrdU incorporation. CD4 T cells were analyzed for intracellular expression of IFN-γ and CXCL10 plus tetanus toxoid-treated DC induced a population of CD4 T cells that expressed significant levels of IFN-γ, while control DC and DC treated with CXCL10 alone did not. CXCL10-treated DC therefore appear to be potent stimulators of T cells.

During DC maturation, members of the MAP kinase family have been shown to be important. Specifically, p38 phosphorylation has been associated with maturation (Arrighi et al., J. Immunol. (2001) 166: 3837-3845), and JNK phosphorylation with IL-12 production. Dephosphorylation of ERK has been associated with survival (Rescigno et al., J. Exp. Med. (1998) 188(11): 2175-2180). To test the effects of CXCL10 on phorphorylation of these MAPK family members, monocytes were cultured for 7 days with GM-CSF and IL-4, and then added CXCL10 for 10 minutes to the resulting immature DC. Cells were immediately placed on ice, then fixed and permeabilized. Intracellular expression of ERK, JNK and p38 were then tested with fluorescent monoclonal antibodies specific for the phosphorylated forms of these proteins. CXCL10 significantly promoted phosphorylation of JNK and p38 while also promoting de-phosphorylation of ERK. Since de-phosphorylation of ERK suggests that CXCL10 may also promote DC survival and LPS has been shown to increase the survival of DC in cultures grown without GM-CSF or IL-4, purified LNDC were cultured with or without CXCL10 in media without the growth factor GM-CSF, and cellular viability tested periodically for 6 days to test whether CXCL10 treatment would increase survival of growth factor-derived DC. CXCL10 significantly increased survival of DC. CXCL10 therefore induces signaling in DC through pathways known to be involved in maturation.

### EXAMPLE 2

### Mouse

Murine CXCL10 was obtained from Peprotech (Rocky Hill, N.J.) and certified by the manufacturer to contain less than 0.1 ng of endotoxin per μg protein. Ovalbumin (OVA, grade VII) was obtained from Sigma-Aldrich (St. Louis, Mo.), and was confirmed to contain less than 0.06 EU/mg protein using the Pyrogen Plus LAL kit (Cambrex, Walkersville, Md.). To select a peptide epitope, the H3L gene sequence of Vaccinia virus encoding the VP35 envelope protein was entered into the SYFPEITHI database. The octameric peptide DSNFFTEL (SEQ ID NO: 1) was chosen as potentially binding to mouse H-2Kb. This peptide was synthesized by Sigma Genosys (The Woodlands, Tex.) at 95% purity, soluble in water, and having a concentration of 0.06 EU/mg protein. Fluorescent monoclonal antibodies to CD3, CD8 and IFN-γ were obtained from Pharmingen (San Diego, Calif.), as was the unlabeled CD32 monoclonal antibody (Fc Block).

The New York City Board of Health Strain (NYCBH) of Vaccinia Virus (provided by the NIH AIDS Reagent Program) was used. Viruses were propagated and titered using standard methods.

For studies using OVA, groups of 10 6-7-week-old C57BL/6 mice (Jackson Laboratory, Bar Harbor, Me.) were injected subcutaneously with 50 μl of either normal saline, 30 μg/kg CXCL10 in normal saline, or 30 μg/kg CXCL10+0.5 mg OVA. All studies were approved by the local animal use committee. Mice were examined daily for side effects including behavioral abnormalities, local inflammation, and anorexia. Blood was sampled from the tail vein for initial tetramer analysis and processed as described herein. Two weeks later, subcutaneous injections were repeated in all animals at the same dosages. On day 28, mice were sacrificed and blood and spleens were tested for the presence of tetramer-positive T cells as below. The entire experiment was performed twice with similar results. For vaccinia infection studies, an adaptation of previously published methods (Owen, D., et al., Nature (1975) 254: 598-9; Tscharke, D and Smith, G, J. Gen. Virol. (1999) 80: 2751-5) to determine immunity to vaccinia was used. Groups of 10 6-7-week-old C57BL/6 mice were injected subcutaneously with 50 μl of normal saline 30 μg/kg CXCL10, 0.3 mg peptide, or 30 μg/kg CXCL10+0.3 mg peptide on day 0. Injections were repeated two weeks later using the same dosages in all animals. On day 28, mice were injected with 10⁵ plaque-forming units (p.f.u.) of vaccinia virus in 50 μl of normal saline intradermally at the base of the tail. This amount was determined in preliminary studies to be sufficient to induce ulcer formation in >95% of non-immune animals. Maximal ulcer formation was scored on a scale of 0-4+, with 0=no lesion, 1+=erythema only, 2+=ulcer present but <2 mm, 3+=ulcer present >2 mm but <1 cm, and 4+=ulcer >1 cm. Ten days after infection, all mice were sacrificed and blood, spleens and tails were harvested for further testing. The experiment was performed twice twice with similar results.

The OVA iTAG H-2 Kb SIINFEKL-PE conjugated tetramer was obtained from Immunomics (San Diego, Calif.) and used according to the protocol of the manufacturer. Whole blood was diluted 1:1 with EDTA/PBS and stained with an experimentally determined optimum concentration of the tetramer, and anti-CD3, CD8 and CD32 (to prevent non-specific binding) antibodies for 1 h at 4° C. in the dark. Blood cells were then lysed with 10 mg/ml saponin (Sigma, St. Louis, Mo.) for 30s, diluted in PBS and immediately analyzed by flow cytometry on a FACSCalibur cytometer (BD Biosciences, San Jose, Calif.), collecting 100,000 events for each sample which typically included 5000 to 10,000 CD3+CD8+ cells. Analysis of flow cytometry results was done with the FlowJo 5.4 software package (Treestar Inc., Ashland, Oreg.) by first gating on CD3+CD8+ cells, then using this gate to plot tetramer staining versus CD8. The cutoff for tetramer-positive cells was determined by gating on CD3+CD8− cells and determining the background staining on this population of cells.

At necropsy, draining lymph nodes were carefully removed and made into single cell suspensions by gentle mechanical disruption. Cells were washed, and then stained with monoclonal antibodies to CD11c, CD8α, MHC II and CD40 for 1 h at 4° C. Cells were then washed, fixed in praformaldehyde (Sigma, St. Louis, Mo.) and analyzed by four-color flow cytometry for the expression of each of the markers. Cells were first plotted for SSC versus CD11c. Positive cells were analyzed for expression of the remaining markers. A total of 10,000 cells were analyzed from ten mice in each group.

Whole blood (50 μl) was cultured in 0.5 ml RPMI 1640 (Cambrex, Walkersville, Md.) +10% fetal bovine serum (FBS, Hyclone, Logan, Utah), along with 100 μg of OVA for 3 days. Monensin (Golgistop, BD Biosciences, San Jose, Calif.) was added to a final concentration of 2 μM on day 3, and 6 h later cells were harvested. After washing, antibodies to CD3, CD8 and CD32 were added and cells were incubated at 4° C. in the dark for 1 h. Cells were washed, placed in Cytofix/Cytoperm (Pharmingen, San Diego, Calif.) to permeabilize cells following the manufacturer's directions. Cells were washed and an anti-IFN-γ monoclonal antibody was added to cells and incubated for 1 h at 4° C. Cells were then washed and analyzed by flow cytometry by first gating on CD3+CD8+ cells, then using this gate to plot IFN-γ versus CD8. Cutoffs for positive cells were set using cells that had not been exposed to monensin as negative controls. The percentage of IFN-γ secreting cells was then calculated using FlowJo. Irrelevant monoclonal antibodies matched to the same isotype as the CD3 and CD8 antibodies served as staining controls (Rat IgG_{2b} and Rat IgG₂ₐ of unknown specificity, respectively from Pharmingen, San Diego, Calif.).

Single-cell suspensions were made of the spleens from animals in each group, and set up in 96 well plates (Coming, Acton Ma.) at 10⁵ cells/well in RPMI 1640 (Cambrex, Walkersville, Md.) +10% FBS (Hyclone, Logan, Utah) and 100 I.U./ml penicillin and 100 μg/ml streptomycin (Cambrex, Walkersville, Md.). Each spleen was tested in triplicate both with and without peptide added at 5 μg/ml. The plate was incubated at 37° C. in 5% CO₂ for 5 days, and the total number of cells present in each well was determined using the CyQuant assay by following the manufacturer's instructions. A stimulation index (S.I.) was calculated as the fluorescence in stimulated wells/fluorescence in unstimulated wells. The experiment was performed twice with similar results.

A fluorometric assay was used to determine antigen-specific cell lysis. Target cells were prepared from the spleens of normal mice by making single cell suspensions of spleens, washing cells, and placing them in RPMI 1640 without serum. The fluorochrome BCECF (Molecular Probes, Eugene, Oreg.) was added to a concentration of 10 μM. The vaccinia-derived peptide was also added at a concentration of 10 μg/ml. Cells were incubated for 30 min at 37° C. in 5% CO₂. Cells were washed and placed in RPMI 1640 with 10% FBS and 100 I.U./ml penicillin and 100 μg/ml streptomycin. Effector T cells were expanded from splenocytes prepared from spleens from each mouse group. Splenocytes were cultured with 10 μg/ml of peptide in RPMI 1640+10% FBS for 5 days. Cells were then washed, placed in RPMI 1640+10% FBS with 100 I.U./ml penicillin and 100 μg/ml streptomycin, and incubated with labeled target cells in ratios of 100:1, 50:1 and 25:1 in 96 well U-bottom plates. Each ratio was set up in triplicate. Wells with target cells alone served as standards for maximum retention of fluorochrome, and wells with target cells lysed with 1% Triton X-100 served as standards for maximum release. The experiment was performed twice with identical results.

Cells were incubated for 4 h at 37° C. in 5% CO₂. The plates were spun down, supernatants removed and cells resuspended in PBS, then transferred to black plates. Fluorescence was then measured, and percent killing was calculated as follows:

$$\frac{\begin{matrix}fluorescence units ( maximum retention ) - \\ fluorescence units ( sample )\end{matrix}}{\begin{matrix}fluorescence ( maximum retention ) - \\ fluorescence ( maximum release )\end{matrix}}$$

Comparisons between groups were analyzed by one-way ANOVA followed by Student's t-test using Prism 4 software (GraphPad, San Diego, Calif.). Categorical tail ulcer data was analyzed using contingency table analysis.

To determine whether parenteral administration of CXCL10 along with an antigen can provoke a specific CD8 T-cell response to that antigen, groups of 10 mice were injected subcutaneously with either: 10, 20, or 30 μg/kg CXCL10 and whole OVA CXCL10 alone, or normal saline. Dosage range was selected by comparison with dosages of other chemokines that demonstrated biological effects.

Two weeks after the first injections, all groups of mice received booster injections using the same concentrations of CXCL10 and/or OVA. Blood samples were drawn from mice at 2 and 4 weeks after the first injections and analyzed for the presence of OVA-specific T cells using tetramers recognizing the OVA-derived peptide SIINFEKL. After the first injections, CXCL10 promoted the development of OVA-specific T cells in 4/10, 5/10 and 5/10 mice injected with OVA and either 10, 20, or 30 μg/kg CXCL10 respectively. After the second injections, a booster response had occurred as now 7/10 mice showed the presence of tetramer-positive cells in the 10 μg/kg CXCL10 group and the overall percentage of tetramer-positive cells was higher (P<0.01 by Student's t-test). Results in the 20 and 30 μg/kg groups were similar. Spleens from each group of animals were analyzed and it was determined that tetramer-positive cells were detectable in spleens from animals that also had tetramer-positive cells in their blood, but at somewhat lower levels (P=0.0031, 0.03, and 0.017 for the 10, 20, and 30 μg/kg groups respectively by Student's t-test). To determine that CD8 T cells induced by CXCL10 and OVA were also functionally reactive, peripheral blood mononuclear cells were incubated with OVA. After incubation, cells were analyzed by flow cytometry for IFN-γ expression. T cells from animals injected with CXCL10 and OVA showed production of IFN-γ, but no IFN-γ-producing cells were found in mice injected with CXCL10 alone or normal saline (P=0.0025 for control versus CXCL10+OVA by Student's t-test). Draining lymph nodes from injected mice were examined for the presence of Langerhans-like cells. Injected antigen has been shown to be taken up and processed by Langerhans cells which mature and present antigen in the draining lymph nodes. If CXCL10 were inducing the maturation of Langerhans cells in the skin, increased numbers of cells would migrate to draining lymph nodes to present antigen. Draining lymph nodes were therefore analyzed for the presence of CD11c+CD8α^{int} MHCII^{high} CD40^{high} mature antigen presenting Langerhans cells. Injection of CXCL10+OVA resulted in a large increase in the percentage of mature Langerhans cells in the draining lymph node. CXCL10 alone also increased the percentage of mature Langerhans cells, but to a lesser degree. In contrast, control mice showed very few mature Langerhans cells. Most Langerhans cells in control animals appeared to be in an immature state. These results demonstrate that CXCL10 acts in vivo to stimulate an antigen-specific CD8 T-cell response.

To determine whether the T cell response to an antigen induced by CXCL10 is sufficient to prevent infection from a viral pathogen, a modification of a vaccinia skin test model was used and mice were injected intradermally with vaccinia virus to determine whether or not they would develop ulcerations. Immune mice either do not develop ulceration or have only slight erythema. Non-immune animals develop localized ulcers after 7-10 days.

Since the VP35 vaccinia coat protein has been shown to be protective in other systems, the SYFPATHI database was used to predict a peptide sequence from this protein that would bind to mouse MHC. Groups of 10 mice were then injected subcutaneously with this peptide (SEQ ID NO: 1) and CXCL10, CXCL10 alone, peptide alone, or normal saline. Injections were repeated 2 weeks later. No side effects were observed in any of the mouse groups. Four weeks after the first injection, mice were challenged with vaccinia virus using intradermal injections at the base of the tail. The quantity of virus injected was found to be sufficient to cause an ulcer in unvaccinated animals in preliminary experiments. Mice injected with both CXCL10 and peptide failed to develop tail ulcerations and were protected from infection (P<0.0001 for CXCL10 + peptide group versus control group using contingency table analysis). Mice injected with CXCL10 or peptide alone developed moderate to severe tail ulcers, as did control mice. These results demonstrate that CXCL10 effectively promotes the development of an immune response to a pathogen-derived peptide antigen that is sufficient to prevent infection.

To determine whether a cell-mediated immune response was generated in animals after injection of CXCL10 and peptide, pathology examination was performed on tail tissue, revealing an inflammatory infiltrate in those mice showing ulcers. Mice injected with CXCL10 and peptide showed only normal tail tissue.

Spleens were removed from all animals and tested for cell-mediated immune response using a lymphocyte proliferation assay. Splenocytes were incubated with or without vaccinia peptide for 5 days, then cell proliferation was quantified by determining the total number of cells present. Splenocytes from animals injected with CXCL10 and vaccinia peptide proliferated and nearly doubled their numbers (S.I.=1.6 versus control=0.8 P <0.01 by Student's t-test), while splenocytes from animals in the CXCL10- or peptide-alone groups did not show proliferation. Virus-specific cytotoxic T lymphocytes (CTL) were then expanded for 5 days, and cytotoxic capability was assessed by adding target splenocytes pulsed with vaccinia peptide and measuring specific lysis. A population of CTL capable of lysing peptide-pulsed splenocytes was present in the spleens of animals injected with CXCL10 and vaccinia peptide, but not present in the other groups (P=0.01 for control versus CXCL10 + peptide at 1:5 ratio by Student's t-test). CXCL10 therefore demonstrated the ability, when administered in conjunction with antigen, to prevent experimental infection with vaccinia virus by inducing a T-cell-mediated immune response.

What is claimed is:

1. A vaccine comprising
an immunogenic amount of at least one antigen chosen from among at least one bacterial antigen, at least one viral antigen, at least one fungal antigen, at least one tumor antigen, or a combination thereof; and
an amount of a CXCR3-binding chemokine effective to stimulate an immune response to the antigen.

2. A vaccine as in claim 1 wherein the at least one viral antigen comprises at least one peptide antigen derived from the amino acid sequence of at least one viral protein.

3. A vaccine as in claim 1 wherein the CXCR3-binding chemokine is IP-10.

4. A vaccine as in claim 1 wherein the CXCR3-binding chemokine is I-TAC.

5. A vaccine as in claim 1 wherein the at least one viral antigen comprises a peptide derived from the amino acid sequence of a vaccinia virus protein.

6. A method for inducing a Th1-type immune response to at least one antigen, the method comprising administering to a subject an immunogenic amount of at least one antigen chosen from among at least one bacterial antigen, at least one viral antigen, at least one fungal antigen, at least one tumor antigen, or a combination thereof; and a therapeutically effective amount of a CXCR3-binding chemokine effective to induce maturation of lymph-node derived dendritic cells.

7. The method of claim 6 wherein administration of the at least one antigen and therapeutically effective amount of a CXCR3-binding chemokine is subcutaneous administration.

8. The method of claim 6 wherein administration of the at least one antigen and therapeutically effective amount of a CXCR3-binding chemokine is parenteral administration.

9. The method of claim 6 wherein administration of the at least one antigen and therapeutically effective amount of a CXCR3-binding chemokine is oral administration.
